(19) United States
(12) Patent Application Publication
(10) Pub. No.: US 2026/0063558 A1
(43) Pub. Date: Mar. 5, 2026

(54) CUP WITH INTEGRATED LATERAL FLOW ASSAY FOR DETECTING CAFFEINE CONTENT

(71) Applicant: SiPhox, Inc., Burlington, MA (US)

(72) Inventor: Diedrik Vermeulen, Boston, MA (US)

(73) Assignee: SiPhox, Inc., Burlington, MA (US)

(21) Appl. No.: 19/320,365

(22) Filed: Sep. 5, 2025

Related U.S. Application Data
(60) Provisional application No. 63/691,015, filed on Sep. 5, 2024.

(51) Int. Cl. G01N 21/78; B01L 3/00; G01N 21/77; G01N 33/14; G01N 33/52

(52) U.S. Cl. CPC G01N 21/78; B01L 3/5023; G01N 33/14; G01N 33/528; B01L 2300/069; B01L 2300/0825; G01N 2021/7759

(57) ABSTRACT

The present disclosure discusses a novel cup designed with an integrated lateral flow assay (LFA) to detect the presence of caffeine in coffee. The LFA is housed in a specialized chamber in a selected position or at the bottom of the cup. This chamber is designed with an opening only at the bottom, which traps air when the cup is filled with liquid, ensuring that only the bottom part of the LFA is wetted by the beverage. This design allows the LFA to function effectively, provided the coffee does not contain milk or other additives that could interfere with the test. The invention includes a transparent window on the cup to allow the user to observe the LFA and determine the caffeine content visually. Additionally, the cup can be modified or retrofitted with LFA chambers that can be attached to existing cups or stand inside them like a straw.

### CROSS-REFERENCE TO RELATED APPLICATIONS

This application claims the benefit of priority of U.S. Provisional Patent Application Ser. No. 63/691,015, filed on Sep. 5, 2024, and titled “CUP WITH INTEGRATED LATERAL FLOW ASSAY FOR DETECTING CAFFEINE CONTENT,” which is incorporated by reference herein in its entirety.

### FIELD OF THE INVENTION

The present disclosure generally relates to beverage containers and, more specifically, to a cup with an integrated lateral flow assay (LFA) for detecting the caffeine content in beverages.

### BACKGROUND

Currently, there is no useful solution for consumers to determine whether their coffee is caffeinated or decaffeinated. Existing methods often require separate devices or tests that are cumbersome and not practical for everyday use.

### SUMMARY OF THE DISCLOSURE

In an aspect, a cup for detecting caffeine content in a beverage includes a lateral flow assay (LFA) integrated within a chamber located at a selected position within the cup, chamber having an opening positioned to allow partial wetting of the LFA when the cup is filled with liquid, trapping air to prevent full immersion, a transparent window positioned on the cup to allow visual observation of the LFA test results

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIGS. 1-4 illustrate exemplary embodiments of the disclosed invention.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Embodiments of this invention provide a convenient and efficient way for consumers to test their coffee's caffeine content directly within the cup from which they are drinking. In some embodiments, a cup is designed with an integrated lateral flow assay (LFA) to detect presence of caffeine in coffee. LFA is housed in a specialized chamber in a selected position or at the bottom of the cup. This chamber is designed with an opening only at the bottom, which traps air when the cup is filled with liquid, ensuring that only the bottom part of the LFA is wetted by the beverage. This design allows the LFA to function effectively. The invention includes a transparent window on the cup to allow the user to observe the LFA and determine the caffeine content visually. Alternatively or additionally, disclosed herein are LFA chambers that can be attached to existing cups or stand inside them like a straw; such chambers may similarly have a portion that allows a strip to be wetted and another that maintains the strip within an air bubble when the chamber is in place and a beverage or other liquid has been poured into the cup.

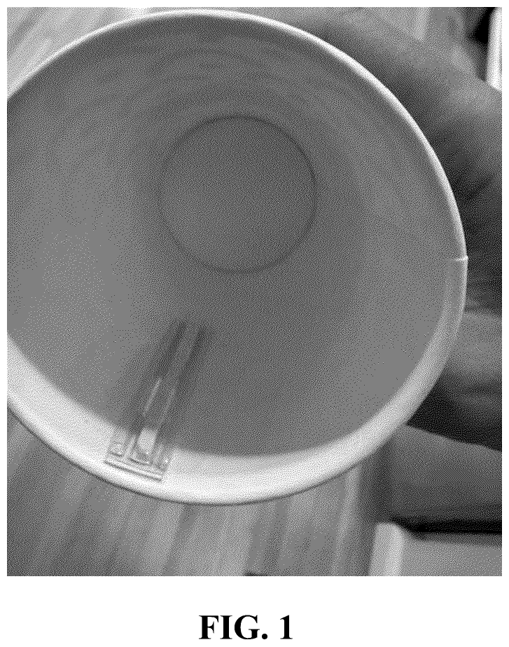

FIG. 1 shows a top-down view into an exemplary embodiment of a cup with an integrated lateral flow assay (LFA). The LFA is positioned inside the cup near the bottom and is housed within a clear, protective chamber. The chamber is attached to the inner wall of the cup, allowing it to remain stable as liquid is poured in. The positioning ensures that the LFA is only partially wetted when the cup is filled, which is essential for the assay's functionality. This setup enables the detection of specific substances, such as caffeine, by limiting exposure to the liquid to only a portion of the assay strip. The image highlights the practical implementation of integrating an LFA into a standard cup for real-time testing.

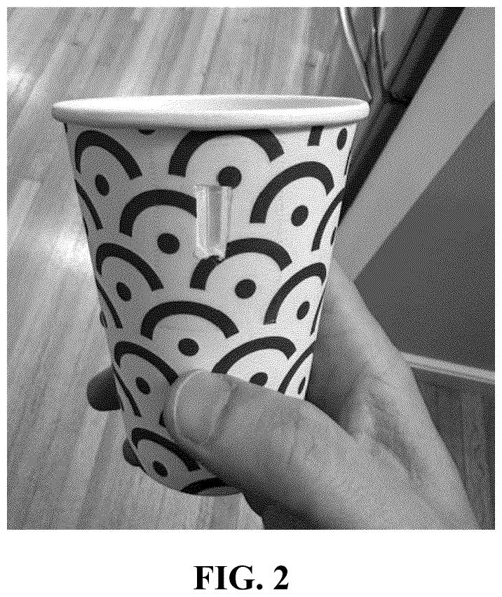

FIG. 2 depicts a side view of an exemplary embodiment of a paper cup, demonstrating the external indication of an integrated lateral flow assay (LFA) within the cup. The cup features a green and white patterned design, and there is a small, rectangular transparent window cut into the side of the cup. This window allows for visual observation of the LFA housed inside the cup without opening or removing any part of the cup. The position of the window aligns with the placement of the LFA inside, enabling users to easily check the assay's results while drinking or after pouring a beverage into the cup. This design provides a practical means for real-time monitoring of the LFA without disrupting the user's experience with the cup.

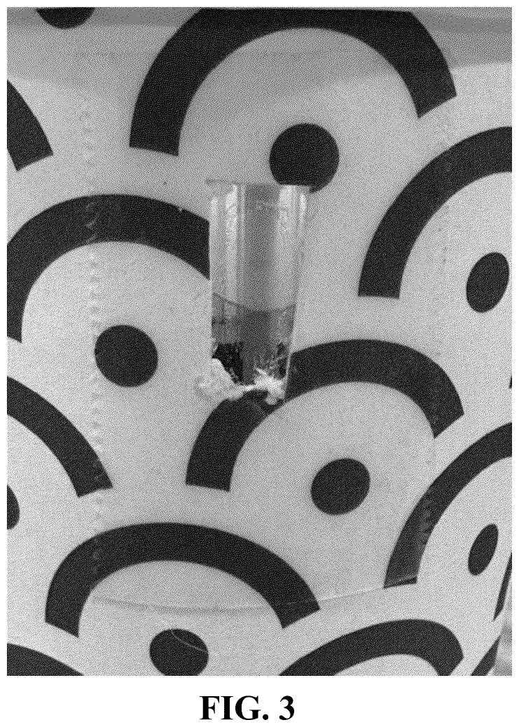

FIG. 3 depicts a close-up view of an exemplary embodiment of a transparent window on the side of a paper cup or cup made of any other suitable material, displaying the integrated lateral flow assay (LFA) while coffee is poured into the cup. The LFA strip, visible through the window, is not fully attached to the side wall of the cup, causing it to blur due to the presence of liquid between the LFA and the wall. This demonstrates a potential design challenge where the partial attachment allows liquid to obscure the LFA lines, making the test results less clear. A solution to this issue would be to fully attach the LFA to the side wall of the cup, preventing any space for liquid to enter and ensuring that the lines remain visible and clear through the window for accurate observation of the assay results.

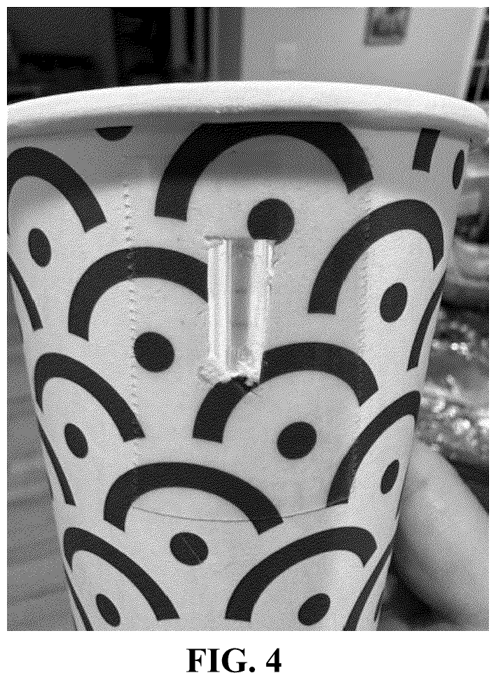

FIG. 4 illustrates an exemplary embodiment of a side view of a paper cup with a transparent window, demonstrating the integrated lateral flow assay (LFA) after coffee has been poured out of the cup. The LFA strip is now clearly visible through the window, displaying two distinct lines. This indicates that the coffee tested was decaffeinated, as the LFA used is a competitive test designed to differentiate between caffeinated and decaffeinated coffee. The clear visibility of the lines suggests that when the liquid is drained from the cup, and the LFA is properly positioned without any obstructive liquid between it and the cup wall, the assay results can be easily observed and interpreted through the window.

Embodiments described herein include a cup integrated with a lateral flow assay (LFA) specifically designed to detect the presence of caffeine in beverages, such as coffee. The LFA is housed within a chamber located at a selected position or at the bottom of the cup, which is strategically designed to have an opening only at the bottom. When the cup is filled with liquid, the air trapped in the chamber ensures that only the bottom part of the LFA is exposed to the liquid, allowing the assay to function correctly.

LFA chamber may be sensitive to the presence of caffeine and is designed to produce visible lines that indicate whether the coffee is caffeinated or decaffeinated. The cup also includes a transparent window for users to observe the test results without needing to open or dismantle any part of the cup.

Furthermore, embodiments as described above may be adapted for various other beverages, such as tea or even spiked drinks, to detect specific substances using appropriate LFA strips. The cup can also be modified to include a mechanism that prevents the entire LFA from being wetted when a person drinks from the cup, preserving the integrity of the test.

Additionally, an attachment can be designed to retrofit existing cups with an LFA chamber that hangs on the side or stands like a straw within the cup, expanding the utility of the invention.

Although in some cases, specific materials have been described for exemplary purposes, embodiments described in this disclosure may be constructed of any suitable transparent and/or non-transparent materials suitable for purposes as described above, including plastic, acrylic, glass, crystal, ceramic, metal, resin, silicone, paper, wood or other plant products, animal products such as without limitation bone or horn, and/or any combination of such materials. LFA chamber may be constructed monolithically with a remainder of a cup and/or may be constructed separately and attached together using any suitable manner for attachment, including adhesion, brazing, welding, soldering, firing in a kiln for heat-based fusion, snapping on using one or more clip or mortise-and-tenon construction, or any other suitable manner of attachment that may occur to a person skilled in the art upon reviewing the entirety of this disclosure.

LFA chamber may contain an assay strip and/or material that permits detection of caffeine, for instance and without limitation as disclosed in U.S. Pat. No. 8,137,984, the entirety of which is incorporated herein by reference.

The above description provides a detailed overview of the preferred embodiment of the invention, with potential modifications and alternative designs also within the scope of the disclosure.

The description herein is provided with the intention to enable a person having ordinary skill in the art to make use of the disclosure. The description herein with connection to the drawings, describes example configurations (e.g. circuit templates) and does not represent all the examples that may be implemented or that are within the scope of the present disclosure. Various modifications to the details provided within the disclosure will be apparent to a person having ordinary skill in the art, and the general principles outlined herein may be applied to other variations without departing from the scope of the disclosure. The term “example” means “serving as an example, illustration, or instance” and not “preferred over other examples”.

Specific details in the detailed description included are provided for the purpose of providing an understanding of the described techniques. These techniques, however, may be practiced with or without these specific details. In some cases, known structures and devices are presented in broad detail to avoid obscuring the concepts of the described examples. Thus, the disclosure is not limited to the examples and designs described herein but is to be conferred the broadest scope consistent with the principles and novel features disclosed herein.

What is claimed is:

1. A cup for detecting caffeine content in a beverage, comprising:
A body forming a receptacle for holding a liquid;
A lateral flow assay (LFA) integrated within a chamber located at a selected position within the cup;
The chamber having an opening positioned to allow partial wetting of the LFA when the cup is filled with liquid, trapping air to prevent full immersion;
A transparent window positioned on the cup to allow visual observation of the LFA test results.

2. The cup of claim 1, wherein the LFA is strategically positioned near the bottom of the cup, allowing it to detect the presence of caffeine specifically in espresso-based coffees prepared in the cup.

3. The cup of claim 1, further comprising:
A mechanism that prevents the entire LFA from being wetted when the cup is tilted or when a user drinks from the cup, maintaining the integrity of the test.

4. A method for detecting caffeine content in a beverage using the cup of claim 1, comprising:
Filling the cup with a beverage such that the liquid contacts only a portion of the LFA through the chamber opening;
Allowing the LFA to react with the beverage to indicate the presence or absence of caffeine;
Observing the results through the transparent window.

5. A detachable LFA assembly for use with a beverage cup, comprising:
An LFA housed within a chamber that can be attached to the side, bottom, or other interior surfaces of the cup;
The chamber having an opening positioned to ensure only partial wetting of the LFA when the cup is filled with liquid.

6. The detachable LFA assembly of claim 5, wherein the assembly includes a closing mechanism to prevent full wetting of the LFA when a user drinks from the cup.\

7. The cup of claim 1, wherein the LFA is configured to detect substances other than caffeine, such as components in tea or alcohol in spiked drinks, by using appropriate test strips.
